(19) United States
(12) Patent Application Publication
(10) Pub. No.: US 2008/0275419 A1
(43) Pub. Date: Nov. 6, 2008

(54) Feminine hygiene product

(76) Inventor: Christine Standfest, Greenwich, CT (US)

Correspondence Address: WIGGIN AND DANA LLP;ATTENTION: PATENT DOCKETING, ONE CENTURY TOWER, P.O. BOX 1832, NEW HAVEN, CT, 06508-1832, US

(21) Appl. No.: 11/799,539

(22) Filed: May 1, 2007

(51) Int. Cl. A61F 13/15

(52) U.S. Cl. 604/385.23

(57) ABSTRACT

One aspect of the invention relates to a sanitary napkin having a fluid permeable top sheet, a fluid impervious bottom sheet and an absorbent layer positioned between the top and bottom sheet that includes an elongated body with a uniform width, having a front section, a rear section, an absorbent hump and at least one absorbent tab.

### BACKGROUND

1. Field

The subject matter of this application relates to a feminine hygiene product, and more particularly to a thin feminine hygiene product with an absorbent hump.

2. Description of the Related Art

Many sanitary napkins are designed to absorb and retain menstrual fluids. In order to obtain the best protection and maximum coverage for the user, sanitary napkins vary in length, thickness, absorbency and style. For example, U.S. Pat. No. 6,171,291 discloses a thin flexible sanitary napkin having a central absorbent hump. The sanitary napkin disclosed in U.S. Pat. No. 6,171,291, is long and wide, which may make it uncomfortable and therefore undesirable to be used by a teenager or a women with a small frame.

Another example of a sanitary napkin is disclosed in U.S. Pat. No. 6,306,123. This patent discloses a sanitary napkin having a stabilizing element, however, the napkin has features that may not maximize comfort, effectiveness and discreteness for petite women and teenage users.

To prevent leakage, it is necessary for the sanitary napkin to conform to the user's body and maintain contact with the user's body as best as possible. Contact between the user and the sanitary napkin often reduces the possibility that liquid will travel around the perimeter of the sanitary napkin and leak.

While sanitary napkins come in a variety of shapes and sizes, to the best of the inventor's knowledge, there is no product that conforms to the body of a petite woman or teenage user. Generally, the longer and thicker, and therefore more effective sanitary napkins, are too cumbersome for a user having a smaller frame. A sanitary napkin designed to fit a petite woman or teenager may increase the protection and comfort of such a wearer.

### BRIEF SUMMARY

One aspect of the present subject matter is a sanitary napkin having a fluid permeable top sheet, a fluid impervious bottom sheet, an absorbent layer positioned between the top sheet and the bottom sheet. The sanitary napkin having an elongated body with generally longitudinal edges wherein the elongated body has a length between about 7 inches and 9 inches and a width between about 1 inch and 3 inches; a front and rear section each having a length between about 1.5 inches and 2.5 inches and a thickness between about 0.1 inch and 0.2 inch; and an absorbent hump positioned between the front and rear sections having a length between about 3 inches and 5 inches and a thickness between about 0.25 inch and 0.75 inch.

Another aspect of the present subject matter is a sanitary napkin having an elongated body with a length between 7 inches and 9 inches, wherein the elongated body has a front section and a rear section each having a thickness between 0.1 inch and 0.2 inch, and an absorbent hump disposed between the front and rear sections, where a thickness of the absorbent hump is at least twice as great as the thickness of the front section and rear section.

Yet another aspect of the present subject matter is a sanitary napkin having a fluid permeable top sheet, a fluid impervious bottom sheet, an absorbent layer positioned between the top sheet and the bottom sheet. The sanitary napkin having an elongated body with generally longitudinal edges where the elongated body has a length of 8 inches and a width of 2 inches, a front and rear section each having a length of 2 inches and a thickness of ⅛ inch, and an absorbent hump positioned between the front and rear sections having a length of 4 inches and a thickness of 0.5 inch.

The details of one or more embodiments are set forth in the accompanying drawings and the description below. Other features, objects and advantages of the invention will be apparent from the description and drawings, and from the claims.

### BRIEF DESCRIPTION OF THE DRAWINGS

For the purpose of illustrating the invention, the drawings show a form of the subject matter that is presently preferred. However it should be understood that the subject matter is not limited to the precise arrangements and instrumentalities shown in the drawings, wherein:

FIG. 1 shows a side view of a sanitary napkin; and

FIG. 2 shows a top view of the sanitary napkin.

Like reference numbers and designations in the various drawings indicated like elements.

### DETAILED DESCRIPTION

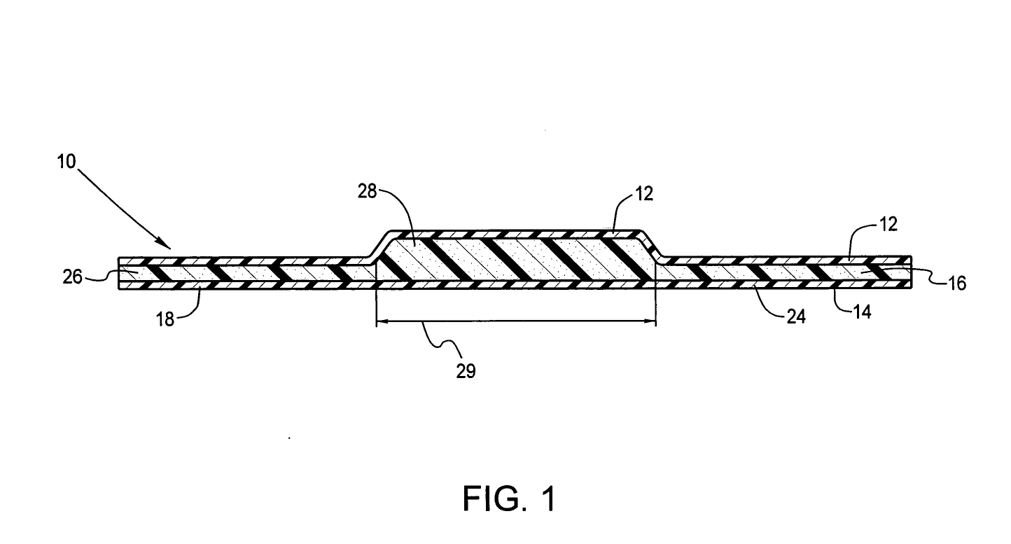

Referring now to the drawings, and particularly to FIG. 1, a sanitary napkin 10 is shown having a fluid permeable top sheet 12, a fluid impervious bottom sheet 14, and an absorbent layer 16. Fluid impervious bottom sheet 14 may be constructed of a thermoplastic material, polyethylene film, or any other material effective to prevent leakage of bodily fluids from sanitary napkin 10. Fluid impervious bottom sheet 14 typically has an adhesive strip (not shown) on one side adapted to adhere to a user's undergarments. This reduces movement of sanitary napkin 10 thereby making it more effective.

Fluid permeable top sheet 12 may be constructed of a material such as a woven material, apertured formed thermoplastic films, apertured plastic films, porous foams, or any other material to maximize liquid absorbency and maintain comfort of the user.

Bodily fluids permeate through fluid permeable top sheet 12 and are trapped in absorbent layer 16. Absorbent layer 16 can be made of a material such as a hydro-gel forming polymeric gelling agent, a cellulose fluff, woodpulp, or any other type of material capable of trapping a liquid. To maximize the effectiveness of sanitary napkin 10, absorbent layer 16 is typically designed to capture and retain liquids.

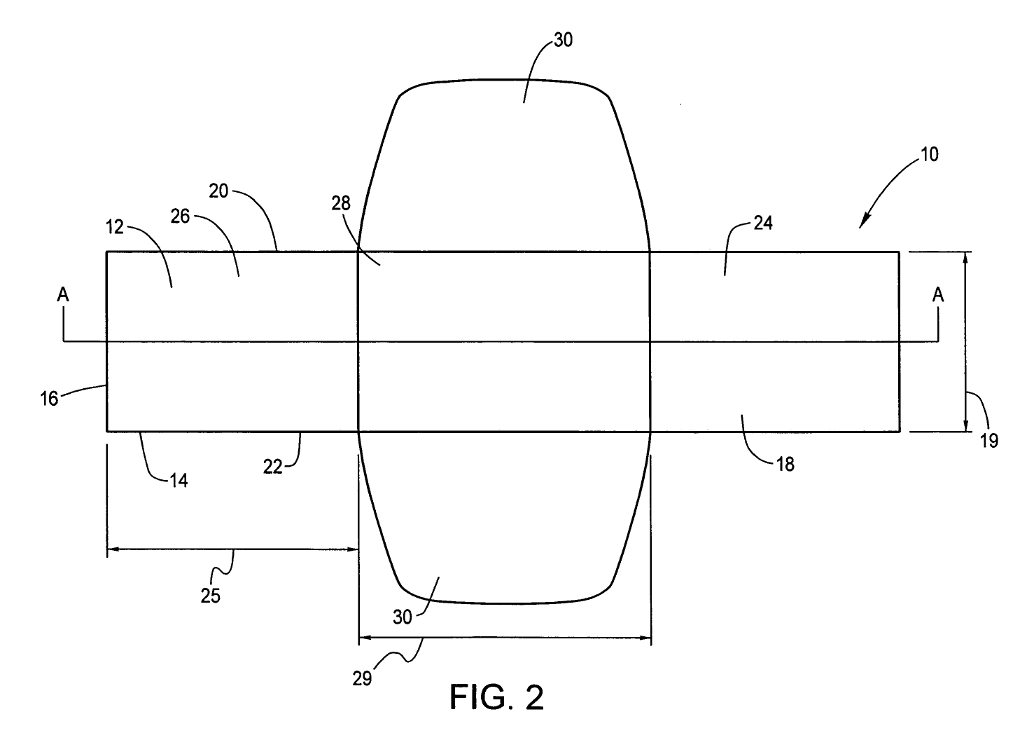

Referring now to FIG. 2, a top view of sanitary napkin 10 is shown. Sanitary napkin 10 has an elongated body 18 with longitudinal edges 20 and 22. Elongated body 18 is typically designed to be generally thin and flexible. In one embodiment, elongated body 18 has a length between about 6 inches and 9 inches In another embodiment, elongated body 18 has a length between 7 inches to 9 inches. In yet another embodiment of sanitary napkin 10, elongated body 18 has a length of about 8 inches. If the length of sanitary napkin 10 exceeds 9 inches, the sanitary napkin becomes less comfortable for the user.

The width 19 of elongated body 18 is uniform along longitudinal edges 20 and 22. The width 19 of elongated body 18 is between about 1 inch and 3 inches. In another embodiment, the width 19 of elongated body 18 is about 2 inches. The narrow width 19 of sanitary napkin 10 makes the sanitary napkin ideal for a teenager or a woman with a small body frame because it is effective, comfortable and is relatively undetectable under clothing. If the dimensions are varied, and the width 19 of elongated body 18 is greater than 3 inches, the comfort and discreteness of sanitary napkin 10 is decreased.

Elongated body 18 has a front section 24 and a rear section 26. Front section 24 and rear section 26 are the thinnest portions of sanitary napkin 10, but still provide protection against leakage of bodily fluids.

Front section 24 and rear section 24 each have a length 25 between about 1 inch and 3 inches. In another example of sanitary napkin 10, front section 24 and rear section 26 each have a length 25 of about 2 inches.

Front section 24 and rear section 26 typically have a thickness in the range of about 0.1 inch to about 0.2 inch. In another embodiment, front section 24 and rear section 26 have a thickness of about ⅛ inch. Front section 24 and rear section 26 are typically designed to hold menstrual fluids from light to moderate flows, i.e., anywhere from 0 to up to 1 ounce. The thin design of front section 24 and rear section 26 allow for added comfort for the user while maintaining an effective absorbency level. The thinness of sanitary napkin 10 will minimize visualization of the sanitary napkin when it is used underneath pants.

Now referring back to FIG. 1, showing a side view of sanitary napkin 10 taken at cross section A-A, an absorbent hump 28 is positioned between front section 24 and rear section 26, and is centered on elongated body 18. Absorbent hump 28 is constructed with fluid permeable top sheet 12, fluid impervious bottom sheet 14 and absorbent layer 16.

Absorbent hump 28 has a length 29 between about 3 inches and about 5 inches. In another embodiment of sanitary napkin 10, absorbent hump 28 has a length 29 between about 3 inches and 4 inches.

To absorb bodily fluids, typically absorbent hump 28 has a thickness between about one fourth of an inch (¼″) to about three fourths of an inch (¾″). Absorbent hump 28 contains approximately twice as much of absorbent layer 16 than front section 24 and rear section 26. Because absorbent hump 28 is centered in the middle of sanitary napkin 10 and is surround by front section 24 and rear section 26 which are both thinner than the absorbent hump, the sanitary napkin can maintain effectiveness while increasing comfort and discreteness.

As shown in FIG. 1, the thickness of absorbent hump 28 is greater than the thickness of front section 24 and rear section 26 to provide for increased fluid retention. Absorbent hump 28 can typically absorb menstrual fluids from light to heavy menstrual flows, i.e., anywhere from 0 to about 3 ounces. Absorbent hump 28 is positioned on sanitary napkin 10 where the greatest amount of fluid would be captured.

Extending away from absorbent hump 28 is at least one absorbent tab 30. Absorbent tab 30 is constructed of a similar material that is fluid absorbing on one side and fluid impervious on the opposite side. Absorbent tab 30 can have adhesive means on the fluid impervious side 14 to wrap around and attach to the users undergarment providing increased stability and greater protection.

Sanitary napkin 10 allows for maximum protection while maintaining comfort and mobility of the user. Absorbent hump 28 is positioned where the maximum of the liquid will be captured and contained. The short length and narrow width of sanitary napkin 10 provide a protective yet more practical option for a user.

One or more embodiments of the subject matter have been described. Nevertheless, it will be understood that various modifications may be made without departing from the spirit and scope of the subject matter described herein. Accordingly, other embodiments are within the scope of the following claims.

What is claimed is:

1. A sanitary napkin having a fluid permeable top sheet, a fluid impervious bottom sheet, an absorbent layer positioned between said top sheet and said bottom sheet; said sanitary napkin comprising:
an elongated body having generally longitudinal edges wherein said elongated body having a length between about 7 inches and 9 inches and a width between about 1 inch and 3 inches;
a front and rear section each having a length between about 1.5 inches and 2.5 inches and a thickness between about 0.1 inch and 0.2 inch; and
an absorbent hump positioned between said front and rear sections having a length between about 3 inches and 5 inches and a thickness between about 0.25 inch and 0.75 inch.

2. A sanitary napkin according to claim 1, wherein said elongated body has a length of 8 inches.

3. A sanitary napkin of claim 1, wherein said elongated body has a width of 2 inches.

4. A sanitary napkin of claim 1, wherein said front and rear sections each has a width of 2 inches.

5. A sanitary napkin of claim 4, wherein said front and rear sections each has a thickness of ⅛ inch.

6. A sanitary napkin of claim 1, wherein said absorbent hump has a length of 4 inches.

7. A sanitary napkin of claim 6, wherein said absorbent hump has a thickness of 0.5 inch.

8. A sanitary napkin of claim 1, further comprising at least one absorbent tab extending away from said absorbent hump.

9. A sanitary napkin comprising:
an elongated body having a length between 7 inches and 9 inches, wherein said elongated body comprises a front section and a rear section each having a thickness between 0.1 inch and 0.2 inch; and
an absorbent hump disposed between said front and rear sections,
wherein a thickness of said absorbent hump is at least twice as great as said thickness of said front section and said rear section.

10. A sanitary napkin according to claim 9, wherein said elongated body has a length of 8 inches.

11. A sanitary napkin of claim 9, wherein said elongated body has a width of 2 inches.

12. A sanitary napkin of claim 9, wherein said front and rear sections each has a width of 2 inches.

13. A sanitary napkin of claim 12, wherein said front and rear sections each has a thickness of ⅛ inch.

14. A sanitary napkin of claim 9, wherein said absorbent hump has a length of 4 inches.

15. A sanitary napkin of claim 14, wherein said absorbent hump has a thickness of 0.5 inch.

16. A sanitary napkin of claim 9, further comprising at least one absorbent tab extending away from said absorbent hump.

17. A sanitary napkin having a fluid permeable top sheet, a fluid impervious bottom sheet, an absorbent layer positioned between said top sheet and said bottom sheet; said sanitary napkin comprising:
an elongated body having generally longitudinal edges wherein said elongated body having a length of 8 inches and a width of 2 inches;
a front and rear section each having a length of 2 inches and a thickness of ⅛ inch; and
an absorbent hump positioned between said front and rear sections having a length of 4 inches and a thickness of 0.5 inch.
